(19) United States
(12) Patent Application Publication
(10) Pub. No.: US 2024/0407741 A1
(43) Pub. Date: Dec. 12, 2024

(54) SYSTEM AND METHOD FOR UPDATING REGISTRATION AND LOCALIZATION DURING SURGICAL NAVIGATION

(71) Applicant: Covidien LP, Mansfield, MA (US)

(72) Inventors: Dany JUNIO, Brookline, MA (US); Steven J. LEVINE, Franklin, MA (US); Jing ZHAO, Superior, CO (US)

(73) Assignee: Covidien LP, Mansfield, MA (US)

(21) Appl. No.: 18/669,674

(22) Filed: May 21, 2024

Related U.S. Application Data
(60) Provisional application No. 63/471,782, filed on Jun. 8, 2023.

(51) Int. Cl. A61B 6/12; A61B 6/00; A61B 6/03

(52) U.S. Cl. CPC A61B 6/12; A61B 6/032; A61B 6/5211

(57) ABSTRACT

A surgical navigation system includes a navigation catheter and a computing device. The computing device is configured to register data detected by a sensor in a luminal network to CT data of the luminal network, and detect when the catheter is located at a node of the luminal network. The computing device is further configured to determine coordinates of the detected node, determine a difference between the determined coordinates of the node and expected coordinates of the node, and determine if the difference between the determined coordinates and the expected coordinates is greater than a predetermined threshold.

### CROSS-REFERENCE TO RELATED APPLICATIONS

This application claims the benefit of U.S. Provisional Patent Application Ser. No. 63/471,782, filed Jun. 8, 2023, the entire content of which is incorporated herein by reference.

### FIELD

This disclosure relates to systems, methods, and devices for dynamically updating registration between sensor data and CT data during a surgical navigation procedure.

### BACKGROUND

There are several commonly applied medical methods, such as endoscopic procedures or minimally invasive procedures, for treating various maladies affecting organs including the liver, brain, heart, lungs, gall bladder, kidneys, and bones. Often, one or more imaging modalities, such as magnetic resonance imaging (MRI), ultrasound imaging, computed tomography (CT), or fluoroscopy are employed by clinicians to identify and navigate to areas of interest within a patient and ultimately a target for biopsy or treatment. In some procedures, pre-operative scans may be utilized for target identification and intraoperative guidance.

For example, an endoscopic approach has proven useful in navigating to areas of interest within a patient, and particularly so for areas within luminal networks of the body such as the lungs. To enable the endoscopic approach, and more particularly the bronchoscopic approach in the lungs, endobronchial navigation systems have been developed that use previously acquired MRI data or CT image data to generate a three-dimensional (3D) rendering, model, or volume of the particular body part such as the lungs. The resulting volume generated from the MRI scan or CT scan may be utilized to create a navigation plan to facilitate the advancement of a navigation catheter (or other suitable medical device) through a bronchoscope and a branch of the bronchus of a patient to an area of interest. A locating or tracking system, such as an electromagnetic (EM) tracking system, may be utilized in conjunction with, for example, CT data, to facilitate guidance of the navigation catheter through the branch of the bronchus to the area of interest. In certain instances, the navigation catheter may be positioned within one of the airways of the branched luminal networks adjacent to, or within, the area of interest to provide access for one or more medical instruments.

However, when navigating inside the lungs according to a pre-operative, CT-based plan, the shape of the lungs is not precisely as it was when the pre-operative CT data was acquired. This mis-match between the CT data and the shape of the lungs during the navigation procedure is known as CT to body divergence. Existing solutions for addressing CT to body divergence require capturing new X-ray images of the patient during the navigation procedure to realign the CT data with the patient's lungs, which is time consuming and exposes the patient to additional and unnecessary radiation.

### SUMMARY

Systems and methods for localization and divergence correction in a surgical navigation procedure are provided.

According to an aspect of this disclosure, a surgical navigation system includes a catheter and a computing device. The catheter is configured to be navigated through a luminal network and to capture images during navigation. The computing device includes a processor and memory storing instructions which, when executed by the processor, cause the computing device to register data, detected by the catheter in the luminal network, to CT data of the luminal network, detect when the catheter is located at a node of the luminal network. The computing device is further configured to determine coordinates of the detected node, determine a difference between the determined coordinates of the node and expected coordinates of the node, and determine if the difference between the determined coordinates and the expected coordinates is greater than a predetermined threshold.

In an aspect, the computing device is configured to determine coordinates of the detected node based on data detected from the catheter.

In an aspect, the computing device is configured to perform image depth sensing to determine a distance between the catheter and the detected node, and to determine coordinates of the detected node based on data detected from the catheter and the determined distance between the catheter and the detected node.

In an aspect, the computing device is configured to update registration of the location data to the CT data of the luminal network if it is determined that the difference between the determined coordinates and the expected coordinates is greater than the predetermined threshold.

In an aspect, the computing device is configured to determine a node corresponding to a location of the catheter if it is determined that the difference between the determined coordinates and the expected coordinates is not greater than the predetermined threshold.

In an aspect, the computing device is configured to detect when the catheter is at the node based on an image analysis of the images captured during navigation.

In an aspect, the computing device is configured to estimate expected changes of the luminal network during navigation, and to determine if changes of the luminal network during navigation differ from the estimated expected changes.

In an aspect, the computing device is configured to analyze a captured image of the node to calculate an angle and a distance between two lumens at the node, and to determine translation and rotation differences between the catheter data and the CT data based on the calculated angle and distance between the two lumens at the node.

In accordance with another aspect of the disclosure, a navigation system includes a catheter, a tracking system operably coupled to the catheter, and a computing device operably coupled to the surgical navigation catheter and the tracking system. The surgical navigation catheter includes a sensor and is configured to navigate along a path through a luminal network. The tracking system is configured to generate location data corresponding to locations of the catheter within the luminal network based on signals received from the location sensor as the surgical navigation catheter is navigated through the luminal network. The computing device includes a processor and memory storing instructions which, when executed by the processor, cause the computing device to register the generated data to CT data of the luminal network, and detect when the catheter is located at a node of the luminal network. The computing device is further configured to determine coordinates of the detected node, determine a difference between the determined coordinates of the node and expected coordinates of the node, and determine if the difference between the determined coordinates and the expected coordinates is greater than a predetermined threshold.

In an aspect, the computing device is configured to determine coordinates of the detected node based on data sensed from the catheter.

In an aspect, the computing device is configured to perform image depth sensing to determine a distance between the catheter and the detected node, and to determine coordinates of the detected node based on data sensed from the catheter and the determined distance between the catheter and the detected node.

In an aspect, the computing device is configured to update registration of the data to the CT data of the luminal network if it is determined that the difference between the determined coordinates and the expected coordinates is greater than the predetermined threshold.

In an aspect, the computing device is configured to determine a node corresponding to a location of the catheter if it is determined that the difference between the determined coordinates and the expected coordinates is not greater than the predetermined threshold.

In an aspect, the computing device is configured to detect when the catheter is at the node based on an image analysis of the images captured during navigation.

In an aspect, the computing device is further configured to estimate expected changes of the luminal network during navigation, and to determine if changes of the luminal network during navigation differ from the estimated expected changes.

In an aspect, the computing device is configured to analyze a captured image of the node to calculate an angle and a distance between two lumens at the node, and to determine translation and rotation differences between the sensor and the CT data based on the calculated angle and distance between the two lumens at the node.

In accordance with another aspect of the disclosure, a method includes registering data, detected by a catheter in a luminal network, to CT data of the luminal network, detecting when the catheter is located at a node of the luminal network, determining coordinates of the detected node, determining a difference between the determined coordinates of the node and expected coordinates of the node, and determining if the difference between the determined coordinates and the expected coordinates is greater than a predetermined threshold.

In an aspect, the method further includes updating registration of the data detected by the catheter to the CT data of the luminal network if it is determined that the difference between the determined coordinates and the expected coordinates is greater than the predetermined threshold.

In an aspect, the method further includes determining a node corresponding to a location of the catheter if it is determined that the difference between the determined coordinates and the expected coordinates is not greater than the predetermined threshold.

In an aspect, the method further includes detecting when the catheter is at the node based on images captured during navigation.

Any of the above aspects and embodiments of this disclosure may be combined without departing from the scope of this disclosure.

### BRIEF DESCRIPTION OF THE DRAWINGS

Objects and features of the system and method disclosed herein will become apparent to those of ordinary skill in the art when descriptions of various embodiments thereof are read with reference to the accompanying drawings, of which:

FIG. 1 is a schematic diagram of a surgical navigation procedure system in accordance with an illustrative aspect of this disclosure;

FIG. 2 is a schematic diagram of a computing device which forms part of the surgical navigation procedure system of FIG. 1 in accordance with an aspect of this disclosure;

FIG. 3 is a flowchart illustrating a method for dynamic localization and registration in accordance with an aspect of this disclosure;

FIG. 4A illustrates a tree representation of a luminal network derived from CT data of the luminal network in accordance with an aspect of this disclosure;

FIG. 4B illustrates an indicator based on location data of a tracked surgical navigation catheter relative to the tree representation of the luminal network of FIG. 4A before dynamic updating of registration between the location data and the CT data is applied in accordance with an aspect of this disclosure;

FIG. 4C illustrates an indicator based on location data of a tracked surgical navigation catheter relative to the tree representation of the luminal network of FIG. 4A after dynamic updating of registration between the location data and the CT data is applied in accordance with an aspect of this disclosure;

FIG. 5A illustrates a three dimensional rendering of a luminal network derived from CT data of the luminal network in accordance with an aspect of this disclosure;

FIG. 5B illustrates an image of a parent node of the luminal network in accordance with an aspect of this disclosure;

FIG. 5C illustrates an image of a child node of the luminal network in accordance with an aspect of this disclosure; and

FIG. 6 illustrates an image of a node within a luminal network in accordance with an aspect of this disclosure.

### DETAILED DESCRIPTION

Although this disclosure will be described in terms of specific illustrative embodiments, it will be readily apparent to those skilled in the art that various modifications, rearrangements, and substitutions may be made without departing from the spirit of this disclosure. The scope of this disclosure is defined by the claims appended hereto.

This disclosure provides a system and method for dynamically updating registration between location data, corresponding to a location of a surgical navigation catheter, and CT data of a luminal network during a surgical navigation procedure. Updating registration is based on the detection of anatomical landmarks during navigation of the surgical navigation catheter through the luminal network. In particular, this disclosure utilizes image data of the luminal network as captured by the surgical navigation catheter to determine the location of the surgical navigation catheter and update the registration of the location data to the CT data.

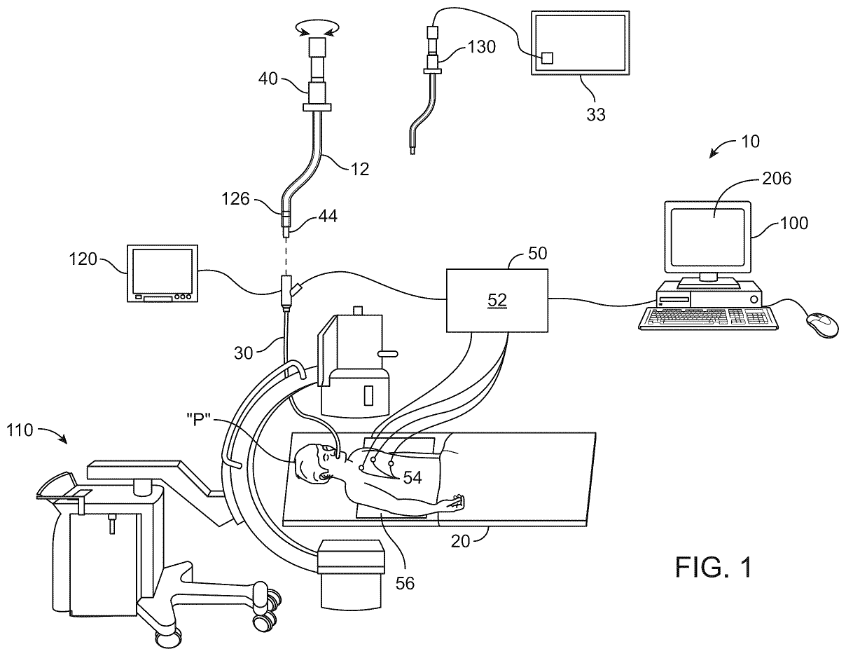

FIG. 1 depicts a surgical navigation system 10 configured for reviewing CT image data to identify one or more targets, planning a pathway to an identified target (planning phase), navigating a catheter 12 (e.g., an extended working channel) of a catheter guide assembly 40 to a target (navigation phase) via a user interface, and confirming placement of the catheter 12 relative to the target. One such system may be an Electromagnetic Navigation (EMN) system such as the ELECTROMAGNETIC NAVIGATION BRONCHOSCOPY® navigation system currently sold by Medtronic plc. The target may be tissue of interest identified by review of the CT image data during the planning phase. Following navigation, a medical instrument, such as a biopsy tool, ablation tool (e.g., ablation device 130), or other tool, may be inserted into the catheter 12 to treat the tissue or obtain a tissue sample from the tissue located at, or proximate to, the target. Although CT data is described, any form of imaging data by any imaging device may be utilized prior to, or during, a procedure.

System 10 generally includes an operating table 20 configured to support the patient “P;” a bronchoscope 30 configured for insertion through patient's “P's” mouth into patient's “P′s” airways; monitoring equipment including a display 120 coupled to bronchoscope 30 (e.g., a video display, for displaying the video images received from the video imaging system of bronchoscope 30); a tracking system 50 including a tracking module 52, a plurality of reference sensors 54 and a transmitter mat 56; and a computing device 100 including software and/or hardware used to facilitate identification of a target, pathway planning to the target, navigation of a medical instrument to the target, and confirmation of placement of the catheter 12, or a suitable device therethrough, relative to the target.

As shown in FIG. 1, catheter 12 is part of a catheter guide assembly 40. In practice, catheter 12 is inserted into bronchoscope 30 for access to a luminal network of patient “P.” Specifically, catheter 12 of catheter guide assembly 40 may be inserted into a working channel of bronchoscope 30 for navigation through a patient's luminal network. A distal portion of the catheter 12 includes a sensor 44 (such as, e.g., a location sensor), and a camera 126. The position and orientation of the sensor 44 within an electromagnetic field, and thus, the distal portion of the catheter 12 relative to a reference coordinate system, can be derived by the tracking system 50. The camera 126 may be any type of sensing device capable of capturing images. As described in greater detail below, computing device 100 analyzes the images captured by the camera 126 to detect when the catheter 12 is located at a bifurcation within the luminal network.

An imaging device 110 capable of acquiring images or video of patient “P” (e.g., fluoroscopic, x-ray, MRI, CT, ultrasonic, etc.) may also be included in this particular aspect of system 10. The image data (e.g., images, series of images, or video) captured by the imaging device 110 may be stored within the imaging device 110 or transmitted to computing device 100 for storage, processing, and display. Additionally, the imaging device 110 may move relative to patient “P” so that images may be acquired from different angles or perspectives relative to patient “P” to create a video from a sweep.

Computing device 100 may be any suitable computing device including a processor and storage medium, wherein the processor is capable of executing instructions stored on the storage medium. The computing device 100 may further include a database configured to store patient data, CT data sets including CT images, fluoroscopic data sets including fluoroscopic images and video, navigation plans, and any other such data. Although not explicitly illustrated, the computing device 100 may include inputs, or may otherwise be configured to receive, CT data sets, fluoroscopic images/video and other data described herein. Additionally, computing device 100 includes a display (e.g., display 206) configured to display graphical user interfaces.

With respect to the planning phase, computing device 100 utilizes previously acquired image data (e.g., CT image data, MRI image data, etc.) for generating and viewing a three-dimensional model or rendering of patient “P's” airways, enables the identification of a target on the three-dimensional model (automatically, semi-automatically, or manually), and allows for determining a pathway through patient “P's” airways to tissue located at and around the target. More specifically, in an aspect, CT images acquired from previous CT scans are processed and assembled into a three-dimensional CT volume, which is then utilized to generate a three-dimensional model of patient “P's” airways. The three-dimensional model may be displayed on a display 206 associated with computing device 100, or in any other suitable fashion. Using computing device 100, various views of the three-dimensional model or enhanced two-dimensional images generated from the three-dimensional model are presented. The enhanced two-dimensional images may possess some three-dimensional capabilities because they are generated from three-dimensional data. The three-dimensional model may be manipulated to facilitate identification of target on the three-dimensional model or two-dimensional images, and selection of a suitable pathway through patient “P's” airways to access tissue located at the target can be made. Once selected, the pathway plan, three-dimensional model, and images derived therefrom, can be saved and exported to a navigation system for use during the navigation phase(s). One such planning software is the ILLUMISITE® planning suite currently sold by Medtronic plc.

With respect to the navigation phase, the tracking system 50 is utilized for performing registration of the images and the pathway for navigation, although other configurations are also contemplated. Tracking system 50 includes a tracking module 52, a plurality of reference sensors 54, and a transmitter mat 56 (including markers if applicable). Tracking system 50 is configured for use with a sensor 44 (such as, e.g., a location sensor) of catheter 12 and may be configured to track, for example, the electromagnetic position thereof within an electromagnetic coordinate system.

Transmitter mat 56 is positioned beneath patient “P.” Transmitter mat 56 generates an electromagnetic field around at least a portion of patient “P” within which the position of a plurality of reference sensors 54 and the sensor 44 can be determined with use of a tracking module 52. One or more of reference sensors 54 are attached to the chest of patient “P.” The six degrees of freedom coordinates of reference sensors 54 are sent to computing device 100 (which includes the appropriate software) where they are used to calculate a patient coordinate frame of reference. Registration, as detailed below, is generally performed to coordinate locations of the three dimensional model and two dimensional images from the planning phase with patient's “P's” airways as observed through the bronchoscope 30, and to allow for the navigation phase to be undertaken with precise knowledge of the location of the sensor 44, even in portions of the airway where the bronchoscope 30 cannot reach.

Initial registration of patient's “P's” location on the transmitter mat 56 is performed by moving sensor 44 through the airways of patient “P.” More specifically, data pertaining to locations of sensor 44, while catheter 12 is moving through the airways, is recorded using transmitter mat 56, reference sensors 54, and tracking module 52. A shape resulting from this location data is compared to an interior geometry of passages of the three dimensional model generated in the planning phase, and a location correlation between the shape and the three dimensional model based on the comparison is determined, e.g., utilizing the software on computing device 100. In addition, the software identifies non-tissue space (e.g., air filled cavities) in the three dimensional model. The software aligns, or registers, an image representing a location of sensor 44 with the three dimensional model and two dimensional images generated from the three dimension model, which are based on the recorded location data and an assumption that sensor 44 remains located in non-tissue space in patient's “P's” airways. Alternatively, a manual registration technique may be employed by navigating the bronchoscope 30 with the sensor 44 to pre-specified locations in the lungs of patient “P”, and manually correlating the images from the bronchoscope to the model data of the three dimensional model.

Though described herein with respect to EMN systems using EM sensors, the instant disclosure is not so limited and may be used in conjunction with flexible sensor, ultrasonic sensors, or without sensors. Additionally, the methods described herein may be used in conjunction with robotic systems such that robotic actuators drive the catheter 12, catheter guide assembly 40 components, or bronchoscope 30 proximate the target.

Following registration of patient “P” to the image data and pathway plan, a user interface is displayed in the navigation software which sets forth the pathway that the catheter 12 is to follow to reach the target.

Once catheter 12 has been successfully navigated proximate the target as depicted on the user interface, the catheter 12 is in place as a guide channel for guiding medical instruments including without limitation, optical systems, ultrasound probes, marker placement tools, biopsy tools, ablation tools (e.g., microwave ablation devices), laser probes, cryogenic probes, sensor probes, and aspirating needles to the target. In an aspect, ablation device 130 is a flexible surgical navigation catheter which is guided through catheter 12 for placement relative to a target and ablation of the target. Ablation device 130 is configured to connect to microwave generator 33 (FIG. 1) which generates and controls the application of microwave energy through the ablation device 130. Microwave generator 33 may be a component of computing device 100 or may be a separate stand-alone component.

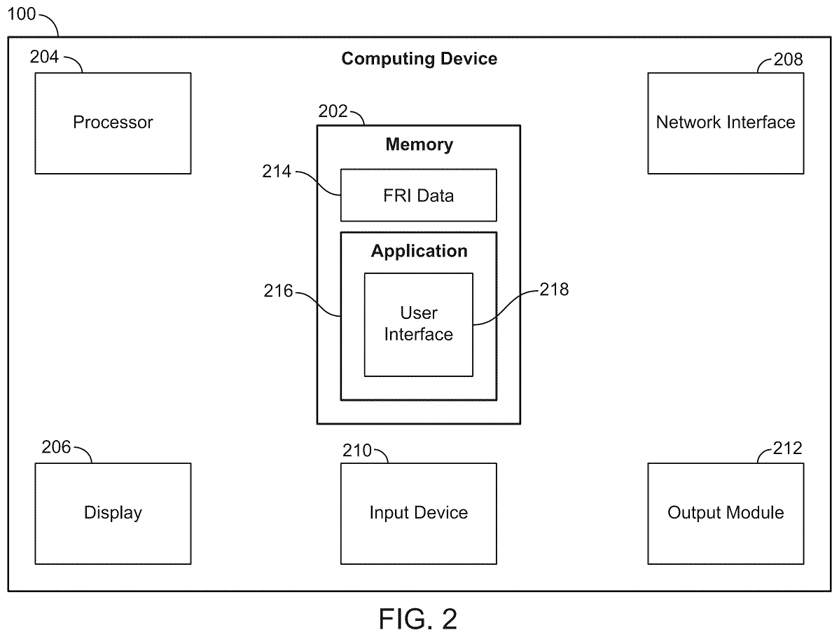

FIG. 2 illustrates a system diagram of computing device 100. Computing device 100 may include memory 202, processor 204, display 206, network interface 208, input device 210, and/or output module 212. Memory 202 includes any non-transitory computer-readable storage media for storing data and/or software that is executable by processor 204 and which controls the operation of computing device 100. In an embodiment, memory 202 may include one or more solid-state storage devices such as flash memory chips. Alternatively or in addition to the one or more solid-state storage devices, memory 202 may include one or more mass storage devices connected to the processor 204 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 204. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium which can be used to store the desired information, and which can be accessed by computing device 100.

Memory 202 may store application 216 and/or functional respiratory imaging data 214 of one or more patients. Application 216 may, when executed by processor 204, cause display 206 to present user interfaces. Processor 204 may be a general-purpose processor, a specialized graphics processing unit (GPU) configured to perform specific graphics processing tasks while freeing up the general-purpose processor to perform other tasks, and/or any number or combination of such processors. Display 206 may be touch sensitive and/or voice activated, enabling display 206 to serve as both an input and output device. Alternatively, a keyboard (not shown), mouse (not shown), or other data input devices may be employed. Network interface 208 may be configured to connect to a network such as a local area network (LAN) consisting of a wired network and/or a wireless network, a wide area network (WAN), a wireless mobile network, a Bluetooth network, and/or the internet. For example, computing device 100 may receive functional respiratory imaging data, DICOM imaging data, computed tomographic (CT) image data, or other imaging data, of a patient from an imaging workstation and/or a server, for example, a hospital server, internet server, or other similar servers, for use during surgical ablation planning. Patient functional respiratory imaging data may also be provided to computing device 100 via a removable memory 202. Computing device 100 may receive updates to its software, for example, application 216, via network interface 208. Computing device 100 may also display notifications on display 206 that a software update is available.

Input device 210 may be any device by means of which a user may interact with computing device 100, such as, for example, a mouse, keyboard, foot pedal, touch screen, and/or voice interface. Output module 212 may include any connectivity port or bus, such as, for example, parallel ports, serial ports, universal serial busses (USB), or any other similar connectivity port known to those skilled in the art.

Application 216 may be one or more software programs stored in memory 202 and executed by processor 204 of computing device 100. During a planning phase, application 216 guides a clinician through a series of steps to identify a target, size the target, size a treatment zone, and/or determine an access route to the target for later use during the procedure phase. In some embodiments, application 216 is loaded on computing devices in an operating room or other facility where surgical procedures are performed, and is used as a plan or map to guide a clinician performing a surgical procedure, but without any feedback from ablation device 130 used in the procedure to indicate where ablation device 130 is located in relation to the plan. In other embodiments, system 10 provides computing device 100 with data regarding the location of ablation device 130 within the body of the patient, such as by EM tracking, which application 216 may then use to indicate on the plan where ablation device 130 is located.

Application 216 may be installed directly on computing device 100, or may be installed on another computer, for example, a central server, and opened on computing device 100 via network interface 208. Application 216 may run natively on computing device 100, as a web-based application, or any other format known to those skilled in the art. In some embodiments, application 216 will be a single software program having all of the features and functionality described in this disclosure. In other embodiments, application 216 may be two or more distinct software programs providing various parts of these features and functionality. For example, application 216 may include one software program for use during the planning phase, and a second software program for use during the procedure phase of the microwave ablation treatment. In such instances, the various software programs forming part of application 216 may be enabled to communicate with each other and/or import and export various settings and parameters relating to the microwave ablation treatment and/or the patient to share information. For example, a treatment plan and any of its components generated by one software program during the planning phase may be stored and exported to be used by a second software program during the procedure phase.

Application 216 communicates with a user interface 218 that generates a user interface for presenting visual interactive features to a clinician, for example, on display 206 and for receiving clinician input, for example, via a user input device. For example, user interface 218 may generate a graphical user interface (GUI) and output the GUI to display 206 for viewing by a clinician.

Computing device 100 is linked to display 120, thus enabling computing device 100 to control the output on display 120 along with the output on display 206. Computing device 100 may control display 120 to display output which is the same as or similar to the output displayed on display 206. For example, the output on display 206 may be mirrored on display 120. Alternatively, computing device 100 may control display 120 to display different output from that displayed on display 206. For example, display 120 may be controlled to display guidance images and information during the microwave ablation procedure, while display 206 is controlled to display other output, such as configuration or status information.

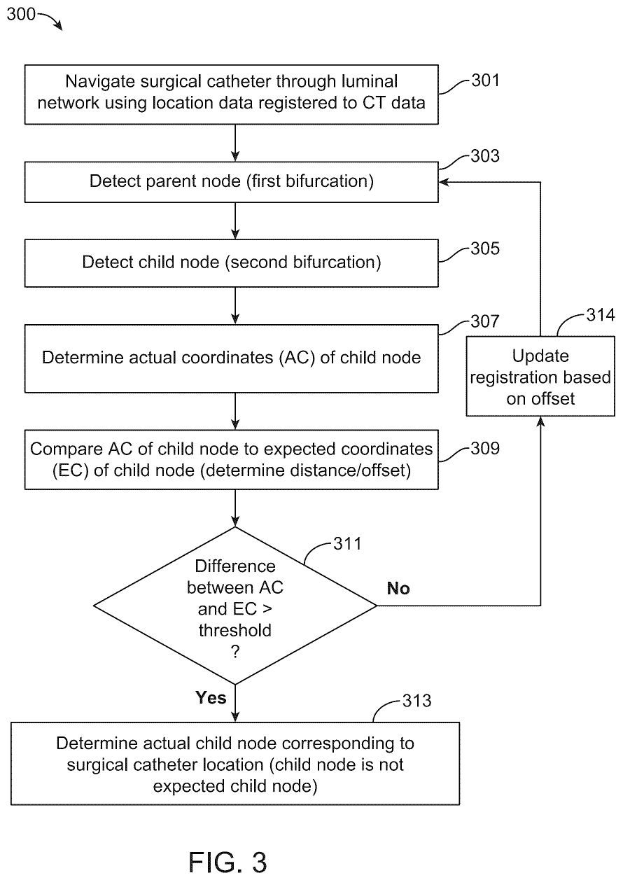

Turning to FIG. 3, a method for dynamically updating registration between location data and CT data during a surgical navigation procedure based on anatomical landmarks within the luminal network is illustrated and described as method 300. Method 300 is described as being executed by computing device 100, but some or all of the steps of method 300 may be implemented by one or more other components of the system 10, alone or in combination. Additionally, although method 300 is illustrated and described as including specific steps, and is described as being carried out in a particular order, it is understood that method 300 may include some or all of the steps described and may be carried out in any order not specifically described.

Method 300 begins at step 301 where a catheter 12 is navigated manually or robotically through a patient's luminal network along a planned path using location data of the catheter 12 that is registered to CT data of the patient's luminal network. In particular, as described above computing device 100 registers location data of the catheter 12 acquired by tracking system 50 to CT data of the luminal network. During navigation, a graphical user interface may be displayed including a CT data rendering (derived from the CT data) and a catheter rendering (derived from the location data of the sensor 44 of the catheter 12 as tracked by the tracking system 50) relative to the CT data rendering based on the registration between the location data and the CT data.

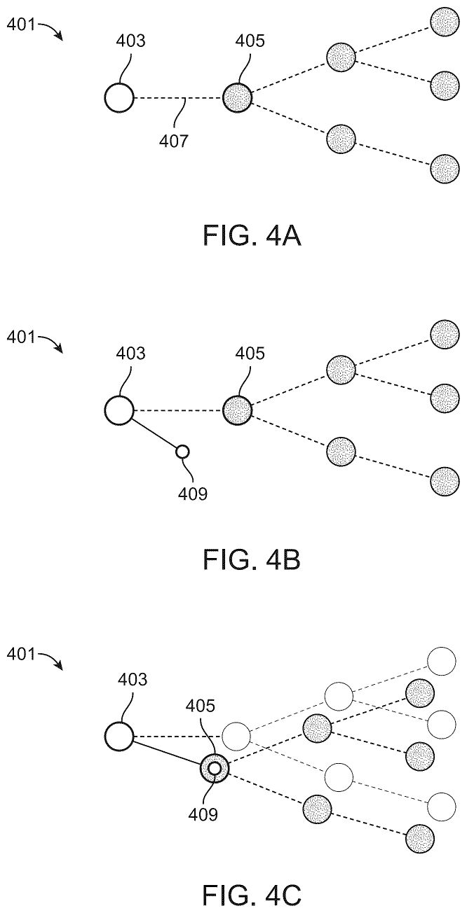

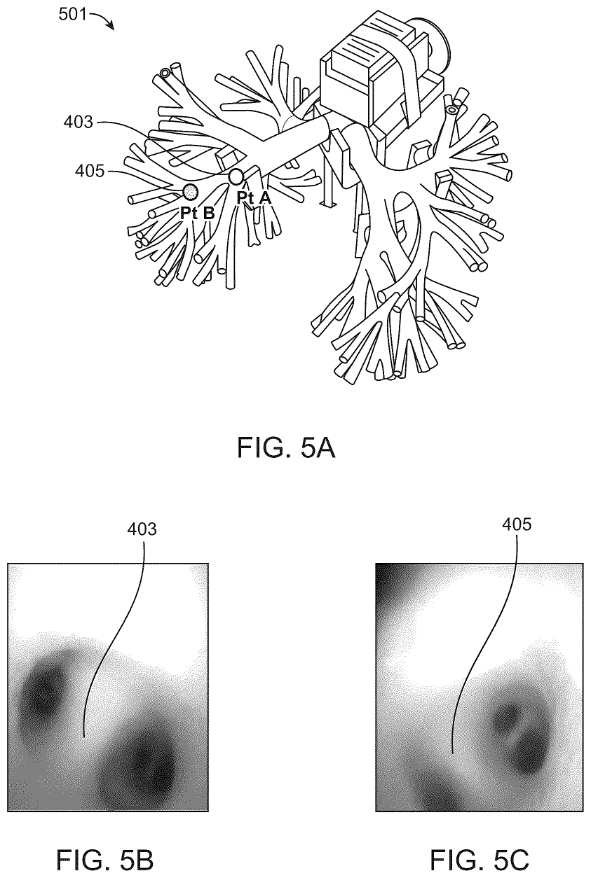

Referring briefly to FIGS. 4A-4C and FIGS. 5A-5C, a tree model 401 (FIGS. 4A-4C) and a three dimensional rendering (FIGS. 5A-5C) of a patient's luminal network are shown, respectively, having a parent node 403 and a child node 405 stemming from the parent node 403 along a piece 407. Each node within the tree model 401 represents an anatomical landmark within the luminal network, such as, for example, a bifurcation within the luminal network. The tree model 401 is uniquely defined by a union of all pieces 407. A catheter indicator 409, representing a location of the catheter 12 derived from the location data of the tracking system 50, is shown positioned relative to the tree model 401 of the patient's luminal network.

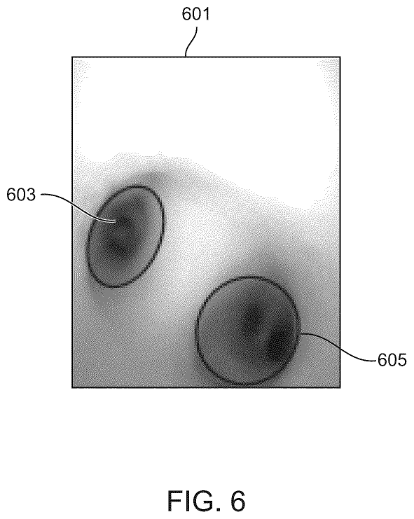

In step 303, computing device 100 detects that the catheter 12 is positioned at (or near) the parent node 403. For example, computing device 100 may analyze the images captured by the camera 126 of the catheter 12 and determine that the image contains distinctive features that correspond to a bifurcation (e.g., the parent node 403) within the luminal network. In an aspect, the computing device 100 generates virtual fly-through images of the luminal network based on the CT data and the images captured by the camera 126 are compared to the virtual fly-through images to detect which bifurcation within the luminal network corresponds to the bifurcation in the captured image. For example, in an aspect, and with reference to FIG. 6, the computing device 100 conducts image recognition on an image 601 captured by the camera 126 based on the number of pixels forming dark areas, and when two dark areas are present within an image (e.g., first dark area 603 and second dark area 605), the computing device 100 determines that a bifurcation is present within the image.

Subsequent to the detection that the catheter 12 is positioned at the parent node 403, the computing device 100 detects that the catheter 12 has been moved to the child node 405 (e.g., along a piece 407 connecting the child node 405 to the parent node 403). Like the detection of the parent node 403, the computing device 100 may analyze the images captured by the camera 126 of the catheter 12 and determine that the image contains distinctive features that correspond to a bifurcation (e.g., the child node 405) within the luminal network and may also factor the distance traveled from the parent node 403 in determining whether a potential image includes a bifurcation corresponding to the child node 405.

In either or both of steps 303 or 305, the computing device 100 may utilize image-based depth sensing techniques to calculate a distance between the catheter 12 and the bifurcation that is depicted in the captured image. The calculated distance between the catheter 12 and the bifurcation that is depicted in the captured image may be factored along with the location data of the tracking system 50 in determining the actual location of the catheter 12 within the luminal network. Additionally, deep learning techniques may be utilized to conduct the image analysis of the captured images for determining whether an image depicts a bifurcation representing a node in the tree model 401. In aspects, computing device 100 may analyze the captured images by detecting lumens within the image and calculating an angle and distance between the lumens detected in the captured image. The distinct angle and distance between the lumens detected in the captured image may be considered when determining the proper translation and rotation factor to apply to the registration update. Additionally, or alternatively, the computing device 100 may factor any physical lumen interaction (e.g., as sensed by the camera 126 and/or by a force sensor connected to the catheter 12) between the catheter 12 and a wall or anatomical feature of the luminal network in steps 303 and/or 305 to detect a parent node 403 and/or a child node 405.

Once the computing device 100 detects that the catheter 12 is located at the child node 405 (based on the image analysis of the images captured by the camera 126 and/or other factors described above), method 300 proceeds to step 307. In step 307, the computing device 100 determines the actual coordinates (AC) of the child node 405 based on the location data derived from the tracking system 50 (e.g., directly corresponding to the location of the catheter indicator 409). In step 309, the actual coordinates of the child node 405 are compared to the expected coordinates (EC) of the child node 405. The expected coordinates of the child node 405 may be determined by the computing device 100 based on the CT data. Step 309 may include determining the distance between the actual coordinates of the child node 405 and the expected coordinates of the child node 405, determining the offset between the two, and/or determining a rotation factor between the two.

In step 311, a determination is made as to whether the difference determined in step 309 exceeds a predetermined threshold which may distinguish between an instance where the location data and the CT data are misaligned due to CT to body divergence and an instance where the catheter 12 has been navigated through a different path and is located at a different node than expected. If in step 311 it is determined that the difference between the actual coordinates of the child node 405 (e.g., the location of the catheter 12) and the expected coordinates of the child node 405 exceeds a predetermined threshold (YES, in step 311), then method 300 proceeds to step 313 where computing device 100 determines the correct (e.g., actual) child node 405 corresponding to the location of the catheter 12 and either repositions the catheter indicator 409 to the correct child node 405 or repositions the correct child node 405 to the catheter indicator 409 to align the location data with the CT data. Step 313 may be carried out by computing device 100 by performing an image analysis of the captured image corresponding to the location of the catheter 12. For example, the captured image may be compared to virtual fly-through images generated from the CT data to detect a match therebetween. In aspects, step 313 additionally includes generating a notification to alert the clinician that the catheter 12 has entered a wrong or unintended branch of the luminal network.

Alternatively, if in step 311 it is determined that the difference between the actual coordinates of the child node 405 (e.g., the location of the catheter 12) and the expected coordinates of the child node 405 does not exceed a predetermined threshold (NO, in step 311), then method 300 proceeds to step 314 where computing device 100 updates the registration between the location data and the CT data based on one or more of a translation or rotation factor offset. In step 314, the offset is applied to the child node 405 and all offspring (e.g., downstream child nodes) of the parent node 403.

In aspects, the computing device 100 may estimate expected changes in the luminal network (e.g., using an atlas or a learning based model) based on previous, similar cases, and the expected changes may be considered in generating the CT data with predictions. In such a case, during a navigation procedure, the computing device 100 may compare the expected changes to the actual changes and determine if the actual changes of the luminal network during navigation differ from the estimated expected changes. If the difference between the expected changes and the actual changes exceeds a predetermined threshold, the computing device 100 may generate a notification to alert the clinician of an abnormality in the procedure.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

What is claimed is:

1. A navigation system comprising:
a catheter configured to be navigated through a luminal network and to capture images during navigation;
a computing device including a processor and memory storing instructions which, when executed by the processor, cause the computing device to:
register data, detected by the catheter in the luminal network, to CT data of the luminal network;
detect when the catheter is located at a node of the luminal network;
determine coordinates of the detected node;
determine a difference between the determined coordinates of the node and expected coordinates of the node; and
determine whether the difference between the determined coordinates and the expected coordinates is greater than a predetermined threshold.

2. The navigation system of claim 1, wherein the computing device is configured to determine coordinates of the detected node based on data detected from the catheter.

3. The navigation system of claim 1, wherein the computing device is configured to:
perform image depth sensing to determine a distance between the catheter and the detected node; and
determine coordinates of the detected node based on data detected from the catheter and the determined distance between the catheter and the detected node.

4. The navigation system of claim 1, wherein the computing device is configured to update registration of the location data to the CT data of the luminal network if it is determined that the difference between the determined coordinates and the expected coordinates is greater than the predetermined threshold.

5. The navigation system of claim 1, wherein the computing device is configured to determine a node corresponding to a location of the catheter if it is determined that the difference between the determined coordinates and the expected coordinates is not greater than the predetermined threshold.

6. The navigation system of claim 1, wherein the computing device is configured to detect when the catheter is at the node based on an image analysis of the images captured during navigation.

7. The navigation system of claim 1, wherein the computing device is further configured to:
estimate expected changes of the luminal network during navigation; and
determine if changes of the luminal network during navigation differ from the estimated expected changes.

8. The navigation system of claim 1, wherein the computing device is configured to:
analyze a captured image of the node to calculate an angle and a distance between two lumens at the node; and
determine translation and rotation differences between the catheter data and the CT data based on the calculated angle and distance between the two lumens at the node.

9. A navigation system comprising:
a catheter configured to navigate along a path through a luminal network, the surgical navigation catheter including a sensor;
a tracking system operably coupled to the catheter and configured to generate data within the luminal network based on data received from the sensor as the catheter is navigated through the luminal network; and
a computing device operably coupled to the catheter and the tracking system, the computing device including a processor and memory storing instructions which, when executed by the processor, cause the computing device to:
register the generated data to CT data of the luminal network;
detect when the catheter is located at a node of the luminal network;
determine coordinates of the detected node;
determine a difference between the determined coordinates of the node and expected coordinates of the node; and
determine if the difference between the determined coordinates and the expected coordinates is greater than a predetermined threshold.

10. The navigation system of claim 9, wherein the computing device is configured to determine coordinates of the detected node based on data sensed from the catheter.

11. The navigation system of claim 9, wherein the computing device is configured to:
perform image depth sensing to determine a distance between the catheter and the detected node; and
determine coordinates of the detected node based on data sensed from the catheter and the determined distance between the catheter and the detected node.

12. The navigation system of claim 9, wherein the computing device is configured to update registration of the data to the CT data of the luminal network if it is determined that the difference between the determined coordinates and the expected coordinates is greater than the predetermined threshold.

13. The navigation system of claim 9, wherein the computing device is configured to determine a node corresponding to a location of the catheter if it is determined that the difference between the determined coordinates and the expected coordinates is not greater than the predetermined threshold.

14. The navigation system of claim 9, wherein the computing device is configured to detect when the catheter is at the node based on an image analysis of the images captured during navigation.

15. The navigation system of claim 9, wherein the computing device is further configured to:
estimate expected changes of the luminal network during navigation; and
determine if changes of the luminal network during navigation differ from the estimated expected changes.

16. The navigation system of claim 9, wherein the computing device is configured to:
analyze a captured image of the node to calculate an angle and a distance between two lumens at the node; and
determine translation and rotation differences between the sensor data and the CT data based on the calculated angle and distance between the two lumens at the node.

17. A method, including:
registering data, detected by a catheter in a luminal network, to CT data of the luminal network;
detecting when the catheter is located at a node of the luminal network;
determining coordinates of the detected node;
determining a difference between the determined coordinates of the node and expected coordinates of the node; and
determining if the difference between the determined coordinates and the expected coordinates is greater than a predetermined threshold.

18. The method of claim 17, further comprising updating registration of the data detected by the catheter to the CT data of the luminal network if it is determined that the difference between the determined coordinates and the expected coordinates is greater than the predetermined threshold.

19. The method of claim 17, further comprising determining a node corresponding to a location of the catheter if it is determined that the difference between the determined coordinates and the expected coordinates is not greater than the predetermined threshold.

20. The method of claim 17. further comprising detecting when the catheter is at the node based on images captured during navigation.
